(19) United States
(12) Patent Application Publication
(10) Pub. No.: US 2007/0185504 A1
(43) Pub. Date: Aug. 9, 2007

(54) Medical clip feeding mechanism

(75) Inventors: Emmanuel Manetakis, Amesbury, MA (US); Peter Aliski, Malden, MA (US)

Correspondence Address: GREENBLUM & BERNSTEIN, P.L.C., 1950 ROLAND CLARKE PLACE, RESTON, VA, 20191, US

(73) Assignee: MICROLINE PENTAX, Inc., Beverly, MA (US)

(21) Appl. No.: 11/210,833

(22) Filed: Aug. 25, 2005

(51) Int. Cl. A61B 17/10

(52) U.S. Cl. 606/142

(57) ABSTRACT

A ladder for use with a clip applying device may include first and second protrusions extending from an end of the ladder along a lateral plane generally coplanar with the ladder or an orthogonal plane generally orthogonal to the ladder, in which the ladder may serially urge a clip outward from the surgical stapler, and the first and second protrusions may abut a cartridge in which the ladder is disposed.

### 1. FIELD OF THE INVENTION

This invention relates to feeding arrangements for medical clip and stapling devices, and more particularly to a surgical stapler for the pinching, stapling, metal suturing or clipping of blood vessels or for the closure of wounds, in which the surgical stapler includes a ladder-type feeding mechanism. The invention also relates to the ladder-type feeding mechanism and cartridge containing the ladder and clips.

### 2. BACKGROUND OF THE INVENTION

Surgical staplers and clips have been used with increasing frequency to replace suturing and/or for closing wounds or to tie-off blood vessels during a surgical procedure or other traumatic medical event. Such surgical staplers generally have a pair of jaws at a distal end of the surgical stapler (that is, the end opposite the handle, trigger, or other actuation assembly for manipulation by the user), which crimp a generally U-shaped clip flatly across the tissues to be tied or sutured. Typically, the clips are arranged in a tube or barrel of the surgical stapler in a sequential manner, the clips being fed serially to the jaws with each actuation of the surgical stapler, for example.

The tools typically in use dispense the clips to the jaws sequentially such that when operated, the distalmost clip is pinched, and then the next adjacent clip immediately therebehind is advanced to the foremost position so that it may be applied next. For example, a clip feeding and dispenser mechanism may use a spring to force the movement of clips in line with an applicator.

Some such surgical staplers include a ladder which pushes a train of such clips forward, one-at-a-time, as the surgical stapler is operated (e.g., serially pushing one of the clips with each squeeze of the actuation assembly). Use of a ladder for pushing the clips is advantageous because the ladder contains several slots (for example, square or rectangular holes) spaced regularly in sequence along the ladder, so that various mechanisms within the surgical stapler can discretely advance the ladder one slot at a time.

However, because the ladder typically has a mass sufficient to slide the ladder along the barrel of the surgical stapler when gravity or inertia acts upon the ladder, there is the possibility that the ladder may slip forward inadvertently while the surgical stapler is in use—for example when the surgeon using the surgical stapler tilts the surgical stapler at an angle steep enough to cause gravity to slide the ladder distalward (that is, toward the distal end) down the barrel. As such, clips may be unintentionally pushed by the ladder and dropped from the surgical stapler, which can complicate the surgical process and which can lead to increased costs.

Furthermore, in some such surgical staplers as shown in FIGS. 23 through 25, a “tail” or “finger” 101 (see FIG. 23) at the proximal end of the ladder 42 (i.e., the end of the ladder closer to the surgeon and not abutting the train of clips; also referred to as the rear) is included which presses upwardly or downwardly (that is, in a direction orthogonal to the plane of the ladder; see the direction of the force F shown in FIG. 25, for example) against a ceiling or floor of a cartridge 30 or the barrel 24 of the surgical stapler in order to generate frictional resistance (see, for example, FIG. 24). However, as shown in FIG. 25, such a “tail” or “finger” 101 (which is exemplified with an end portion 102) generates a retrograde force orthogonal to the flat plane of the ladder 42, which can warp or bend the ladder and cause undesirable effects—as shown, for example, in FIG. 25, in which the arrow F illustrates the upward force of the tail 101 exerted against the cartridge 30 via a rounded portion 102, and the resultantwarping of the ladder 42. It is noted that the tail 101 may alternatively exert a downward force against the cartridge with similar effect, where the cartridge 30 opens downwardly instead of upwardly, for example.

Hereinafter, an example of a stapling gun 14 which may be outfitted with a ladder as described above is discussed, in reference to FIGS. 1 through 3. Referring now to FIG. 1, for example, a clip or staple advancing and feeding arrangement 10 for providing clips or staples to the jaws 12 (which are one example of a clip applicator, inter alia) of a stapling gun 14 (also referred to, for example, as a “clip applying device,” “medical stapler” or “staple clip gun,” inter alia) is shown. The staple clip gun 14 includes a handle 16 at a first end (also referred to hereinafter as the “proximal end”) thereof, for holding and actuating the clip stapling arrangement 10. The handle 16 includes a housing 18 and a trigger mechanism 20 for actuating the jaws 12 and the clip feeding mechanism 10.

The housing 18 has an opening 22 through which a proximal end 25 of a barrel 24 is supported. The barrel 24 also has a distal end 26 (in other words, the end farther from the handle 16 than the proximal end) where the jaws 12, utilized for feeding and pinching the clips or staples 28, are located. A generally U-shaped or C-shaped frame member 40 (see, for example, FIGS. 2 through 4) extends through the length of the interior of the barrel 24, and may have a central trench or hole running generally along a center of the frame member 40. The frame member 40 typically has a plurality of bridges (not shown) spaced along its elongated length. A jaw control rod (not shown), moveable proximally and distally, is supportively arranged on the bridges to provide the pinching movement to the jaws 12 at the distal end 26 of the barrel 24.

The frame member 40 has several portions along its length on its lowermost side, having elongated slots 38 therein, as may be seen in FIGS. 2 and 4. The distalmost slot 38′ is arranged near the distal end 26 of the barrel 24, and a mid-slot (not shown) may be arranged along a mid portion of the frame member's length.

The barrel 24 may be arranged to receive a clip cartridge 30 at its rearmost opening 22 at the housing 18 in the handle 16 (as an alternative, for example, the clip cartridge 30 may be inserted into the barrel or from the distal end, inter alia). The frame member 40 may be arranged to receive the generally U-shaped or C-shaped (cross-section) clip or staple cartridge 30 which may include one or more medical clips or staples 28 and an elongated ladder 42, as illustrated in FIG. 4, for example. The clips 28 may be generally U-shaped, for example, and may have leg members 29 which extend distally away from the housing 18; alternatively, the clips 28 may have any appropriate shape.

The clips 28 and the elongated ladder 42 may be slidably movable within the generally U-shaped frame member 40 and/or the cartridge 30, which provides their enclosure in the package. The elongated ladder 42 may have side rails 43 extending on each side of the ladder 42 and extending along the length of the ladder 42, as well as cross rails 45 connected orthogonally between the side rails 45 and forming a stepladder-like structure including a plurality of elongated slots or holes 44 of generally rectangular shape arranged longitudinally down the middle portion of the ladder 42, as illustrated in FIGS. 23 and 24, for example (although as an alternative, the slots or holes 44 may be of any suitable shape such as, for example, an oval, circular, or rounded outline; further, the slots or holes 44 may be offset from the center of the ladder 42, or may be indentations rather than fully penetrating holes, for example).

The ladder 42 has a distal end 48, which abuts the last (i.e., proximalmost) clip 28 within the cartridge 30. As an example, about twenty clips or staples 28 may be held seriatim in the cartridge 30; alternatively, however, the cartridge may hold just one clip, or it may hold any particular number of clips which suitably fit within the space available within the frame member 40. Each clip 28 preferably abuts its immediate neighbors and may be disposed in a pushing arrangement with its adjacent distal clip 28, for example.

An elongated cycling clip feeder member or bar 50 may be arranged within the barrel 24 and may be supported beneath the frame member 40 therewithin. The clip feeder member or bar 50 may have a distalmost finger 52 of ramp-like configuration, having a short pin 54 extending from each transverse side thereof, as may be seen in FIGS. 1 through 3. The cycling clip feeder bar 50 may also have a proximal finger 60 extending upwardly in a spaced relation to the elongated slot location in the lowermost side of the frame member 40.

The elongated clip feeder bar 50 is movable distally and proximally according to the actuation of the trigger mechanism 20 within the handle 16 of the clip device 14. The proximate finger 60 also has a pin (not shown), extending transversely from each side thereof in a manner similar to that of the distalmost finger 52. The pins 54 on the fingers 52 and 60 cam the fingers 52 and 60 out of engagement with the clips 28 through sliding engagement with the ramps 66 and 68 adjacent the slots 38 in the frame member 40, as the feeder bar 50 is drawn proximally.

The squeezing of the trigger mechanism 20 cycles the longitudinal distal advancement of the elongated clip feeder bar 50 relative to the frame member 40. Release of that trigger simultaneously advances the proximal finger with the distal finger 52, and the proximal finger 60 is permitted by the upward spring action thereof, to enter one of the holes 44 of the elongated ladder 42 corresponding to the location of the slot opening (also referred to as a central trench) in the lower mid portion of the cartridge 30 and frame member 40, as exemplified in FIG. 4, and shown in FIG. 3. The same advance of the clip feeder bar 50 may effect advancement of its distalmost finger 52. As the distalmost finger 52 advances, it engages the backside of the distalmost clip 28, as represented in FIG. 6, to push thereon and effect its entry between the pincher jaws. The trigger mechanism 20 effects the squeezing together of the open pincher jaws 12 to crimp the clip or staple 28.

Release of the trigger mechanism 20 opens the jaws 12 (which are one example of a clip applicator, inter alia) and advances the proximal finger 60, which has by then traveled the length of the rectangular hole 44 in the ladder 42 at its location adjacent its opening in the frame member 40. As the proximal finger 60 engages the distal end of that rectangular hole 44 in the ladder 42, it then begins to push the ladder 42 distally a spaced distance, to push upon the entire series of clips 28 within the cartridge 30 and thus effecting delivery of the next available staple or clip 28 to its “stand-by” position at the distal end 26 of the barrel 24 after the former end or distalmost clip 28 has been pushed between the jaws 12 by the distalmost finger 52.

Movement of the trigger mechanism 20 effects rearward or proximate cycle of movement of the clip feeder bar 50, and the side pins 54 of each respective finger 52 and 60 engaging the lower side of the ramps 66 and 68 adjacent the slot 38 on the lower side of the frame member 40, so as to bias the fingers 52 and 60 downward and out of the way of the respective clips 28 and ladder openings or holes 44 respectively adjacent thereto as the feeder bar 50 cycles rearwardly completing a generally oval path. The clip finger bar 50 is then returned to its proximalmost location with the pins 54 riding under the feeder guide plate 66, to await a further actuation of the trigger mechanism 20, which would recycle the entire clip cartridge 20 and feeder bar mechanisms 50 accordingly.

However, as discussed above, use of a such a clip applying device with inadequate sliding movement control includes the risk that a staple or clip may be inadvertently ejected or dropped from the distal end of the barrel, because the ladder (which may have a not-insignificant mass and thus be susceptible to sliding due to gravity or inertia) can slide within the frame of the barrel when gravity or inertia are exerted on the ladder, such as can occur when the user of the clip applying device causes the barrel to tilt downward or when the clip applying device is rapidly moved, for example. When the ladder slides because of such a force, it may press against the clips to the extent that one or more of the clips may unintentionally emerge from the distal tip of the barrel, for example. Furthermore, the use of a “tail” or “finger” on the ladder which presses downwardly against the cartridge and/or the frame of the barrel can generate undesirably warping of the ladder.

### SUMMARY OF THE INVENTION

Accordingly, it is a feature of the present invention to generate appropriate resistance to sliding movement of a ladder in a surgical stapler and/or clip applying device.

According to an aspect of the present invention, a clip applying device may include a barrel having a first end and a second end, a clip applicator affixed to the second end of the barrel and which can apply a clip during a surgical procedure, an actuation assembly attached to the first end of the barrel and which can actuate the ladder and clip applicator when operated by a user, a cartridge disposed within the barrel extending generally from the first end of the barrel to the second end of the barrel, and a ladder slidably disposed within the cartridge and including a first protrusion extending laterally outward from a central axis of the ladder.

The clip applying device may also include a second protrusion extending laterally outward from a central axis of the ladder in a direction generally opposite from the first protrusion; the cartridge may have a cross-section generally similar to a letter ‘C’ and include first and second inner side surfaces, and the first and second protrusions of the ladder may frictionally abut the first and second inner side surfaces, respectively; the ladder may be substantially flat when disposed in the cartridge; the ladder may include a clip abutment portion having the first protrusion and which can abut a first clip nearest the actuation assembly among a plurality of clips, and in which the first clip has a shape that can conform to the clip abutment portion of the ladder; the cartridge may be insertable into and removable from the clip applying device, may be capable of housing the ladder therein, and may be capable of containing two or more clips, in which the cartridge can be inserted into the first end of the barrel; and/or the first protrusion may extend from at least one of a distal end of the ladder farthest from the actuation assembly, a proximal end of the ladder nearest to the actuation assembly, and/or a point between the proximal and distal ends.

In accordance with another aspect of the present invention, a clip applying device may include a barrel having a first end and a second end, a clip applicator affixed to the second end of the barrel and which can apply a clip during a surgical procedure, an actuation assembly attached to the first end of the barrel and which can actuate the ladder and clip applicator when operated by a user, a cartridge disposed within the barrel extending generally from the first end of the barrel to the second end of the barrel, and a ladder slidably disposed within the cartridge and including a side rail extending longitudinally within the cartridge, a first protrusion extending from a top of the rail and a second protrusion extending from a bottom of the rail at a position opposite the first protrusion, in which the first and second protrusions can abut the cartridge.

In regards the above-noted clip applying device, the cartridge may have a cross-section generally similar to a letter ‘C’ and include a bottom inner surface and a top inner surface generally parallel to the bottom surface, and the first and second protrusions of the ladder may frictionally abut the top inner surface and the bottom inner surface, respectively; the first protrusion may exert a downward force and the second protrusion may exert an upward force, in which the upward force and the downward force impart generally no net torque to the ladder; the ladder may be substantially flat when disposed in the cartridge; the clip applying device may further include a clip abutment portion which can abut a first clip nearest the actuation assembly among a plurality of clips, and in which the first clip may have a shape which can conform to the clip abutment portion of the ladder; the cartridge may be insertable into and removable from the clip applying device, may be capable of housing the ladder therein, and may be capable of containing two or more clips and of being inserted into the first end of the barrel; and/or the first and second protrusions may extend from a distal end of the ladder farthest from the actuation assembly, a proximal end of the ladder nearest to the actuation assembly, and/or a point between the proximal and distal ends.

In accordance with yet another aspect of the present invention, a ladder for use with a clip applying device may include first and second protrusions extending from an end of the ladder along a lateral plane generally coplanar with the ladder or an orthogonal plane generally orthogonal to the ladder, in which the ladder which can serially urge a clip outward from the clip applying device, and in which the first and second protrusions may abut a cartridge in which the ladder is disposed.

In regards to the above-noted cartridge, the cartridge may engage a cartridge within a barrel of the clip applying device, in which the cartridge has a cross-section generally similar to a letter ‘C’ and includes first and second inner side surfaces, and in which the first and second protrusions of the ladder frictionally abut the first and second side surfaces, respectively; the ladder may include a plastic material; the cartridge may engage a cartridge within a barrel of the clip applying device, in which the cartridge has a cross-section generally similar to a letter ‘C’ and includes an inner bottom surface and an inner upper surface, and in which the first and second protrusions of the ladder include vertical risers which can frictionally abut the inner bottom surface and the inner upper surface, respectively; the ladder may include a clip abutment portion including the first and second protrusions, in which the clips have a generally parabolic shape which can conform to the clip abutment portion of the ladder; and/or the cartridge may further include third and fourth protrusions extending from an end of the ladder generally opposite the first and second protrusions and which can abut the cartridge in which the ladder is disposed.

It is noted that although a surgical or medical clip applicator which is reusable and receives a cartridge to reload clips has been described as an example, the present invention is not limited to a reusable clip applying device but may also be used in a single-use clip applying device or any other suitable type of clip applying device, for example.

Other exemplary embodiments and advantages of the present invention may be ascertained by reviewing the present disclosure and the accompanying drawings, and the above description should not be considered to limit the scope of the present application.

### BRIEF DESCRIPTION OF THE DRAWINGS

The present invention is further described in the detailed description which follows, in reference to the noted drawings, by way of non-limiting examples of certain embodiments of the present invention, in which the numerals represent like elements throughout the several views of the drawings, and in which:

FIG. 1 is a partially cut-away side view of a surgical stapler;

FIG. 2 is a cross-sectional view of the barrel of the surgical stapler shown in FIG. 1, taken along line X-X;

FIG. 3 is an enlarged partially cut-away detail view of the distal end of the surgical stapler shown in FIG. 1;

FIG. 4 is a partially cut-away oblique view of a ladder in a cartridge engaged with a frame according to a first embodiment of the present invention;

FIG. 5 is a plan view of a ladder according to the first embodiment;

FIG. 6 is a partially cut-away plan view of the ladder shown in FIG. 5 slidably engaged in a cartridge;

FIG. 7 is an enlarged plan view of the proximal end of the ladder shown in FIG. 5;

FIG. 8 is an oblique view of the proximal end of the ladder shown in FIG. 5;

FIG. 9 is an oblique view of a ladder having proximal protrusions according to the first embodiment;

FIG. 10 is an oblique view of the ladder shown in FIG. 9 slidably engaged in a cartridge, with an upward perspective viewed from generally below the central trench of the cartridge;

FIG. 11 is a cut-away side view of the ladder slidably engaged in the cartridge, as shown in FIG. 10, taken along line A-A;

FIG. 12 is an oblique view of a ladder according to a second embodiment of the present invention having distal protrusions;

FIG. 13 is an oblique view of the ladder shown in FIG. 12 slidably engaged in a cartridge, with an upward perspective viewed from generally below the central trench of the cartridge;

FIG. 14 is a cut-away side view of the ladder slidably engaged in the frame, as shown in FIG. 13, taken along line B-B;

FIG. 15 is an oblique view of a ladder having generally stubby distal protrusions according to a third embodiment of the present invention;

FIG. 16 is an oblique view of a second example of a ladder according to the third embodiment;

FIG. 17 is an oblique view of a ladder having a conformally shaped abutment portion according to a fourth embodiment of the present invention and a corresponding clip;

FIG. 18 is an oblique view of a second example of a ladder and clip according to the fourth embodiment;

FIG. 19 is an oblique view of a ladder having orthogonal vertical risers according to a fifth embodiment of the present invention;

FIG. 20 is a side view of the ladder shown in FIG. 19;

FIG. 21 is a front view of the ladder shown in FIG. 19;

FIG. 22 is a cutaway front view of the ladder shown in FIG. 19 slidably engaged in a cartridge in which the orthogonal vertical risers are cross-positioned and abut the ceiling and flanges of the cartridge;

FIG. 23 is an oblique view of a conventional ladder having a tail or finger at the proximal end thereof;

FIG. 24 is an oblique view of the ladder shown in FIG. 23 slidably engaged in a cartridge, with an upward perspective viewed from generally below the central trench of the cartridge; and

FIG. 25 is a cut-away side view of the ladder shown in FIG. 24 within the cartridge, showing the warping of the ladder caused by the upward force exerted by the tail, taken along line C-C.

### DETAILED DESCRIPTION OF THE PREFERRED EMBODIMENTS

The particulars shown herein are by way of example and for purposes of illustrative discussion of the embodiments of the present invention only, and are presented for providing what is believed to be the most useful and readily understood description of the principles and conceptual aspects of the present invention. In this regard, no particular attempt is made to show structural details of the present invention in more detail than is necessary for the fundamental understanding of the present invention, the description taken with the drawings making apparent to those skilled in the art how the several forms of the present invention may be embodied in practice, although certain examples may be described in fuller detail in order to convey certain aspects of embodiments of the present invention.

Furthermore, although some embodiments of the present invention may include certain features generally similar to those discussed above with regard to the Background of the Invention, the present invention is not limited to the examples discussed therein but rather encompasses all configurations and/or designs which embody the present invention, such as (but not limited to) the exemplary embodiments discussed below, for example.

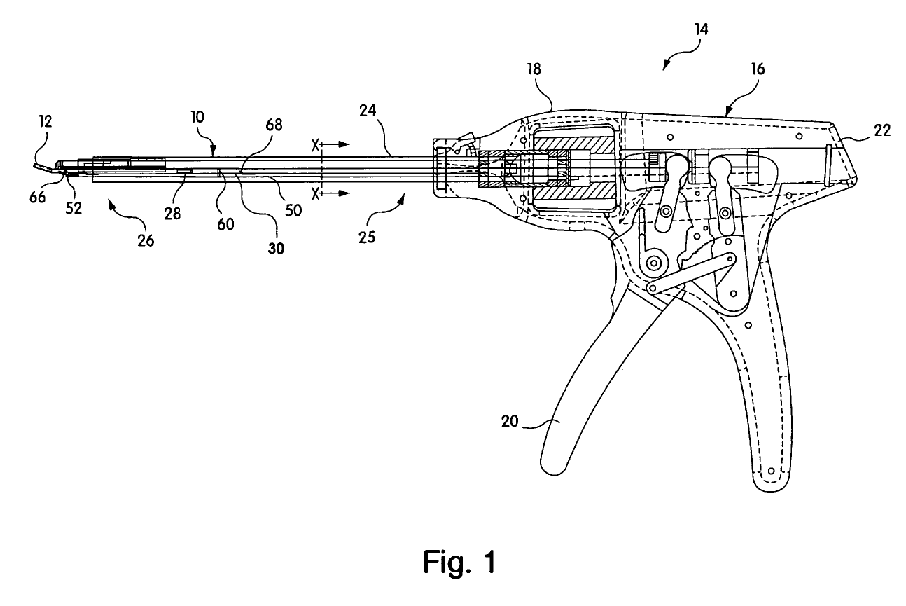

In accordance with a first embodiment of the present invention, a stapling gun 14 (as shown in FIG. 1, for example) may include a clip or staple advancing and feeding arrangement 10 for providing clips or staples to the jaws 12 of the stapling gun 14, for example. A ladder 42 may be generally positioned within a barrel 24, which has a distal end 26 generally proximal to or aligned toward the distal end of the stapling gun 14 that includes the jaws 12, for example, and a proximal end generally proximal to, aligned toward, an/dor abutting the housing 18 of the handle 16.

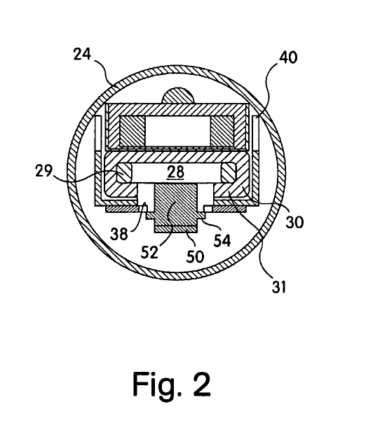

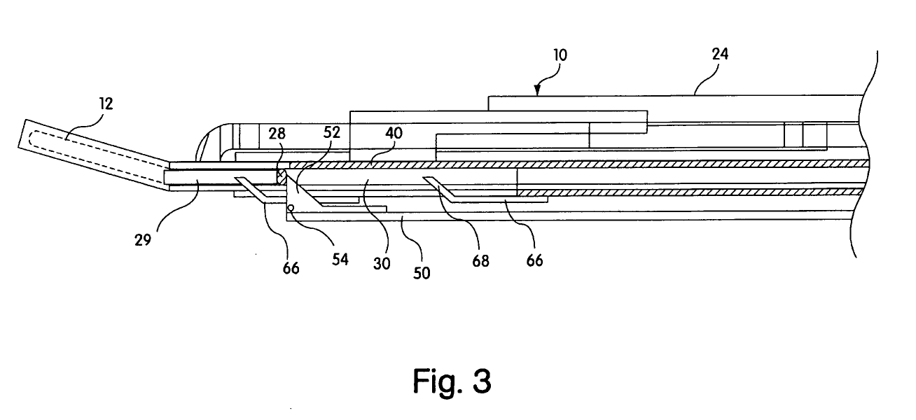

The barrel 24 may be flexible or, alternatively, may be rigid, for example, or may have any appropriate properties of flexibility and/or rigidity. Also, the barrel 24 may have a cross-section of any suitable shape or form. As illustrated in FIG. 2, in a non-limiting example, at least one embodiment of the present invention may include a barrel 24 with a generally circular and/or elliptical cross-section—however, the barrel 24 may alternatively have any of, inter alia, a generally flat, elongated, helical, sinusoidal, triangular, rectangular, square, hexagonal, and/or complex cross-section; and/or the cross-section of the barrel 24 may be different at various portions along the length of the barrel 24, for example (the examples of cross-sections noted above are not intended to limit the cross-section of the barrel 24 according to any embodiment or embodiments of the present invention thereto, but are described as non-limiting examples of cross-sections the barrel 24 may have).

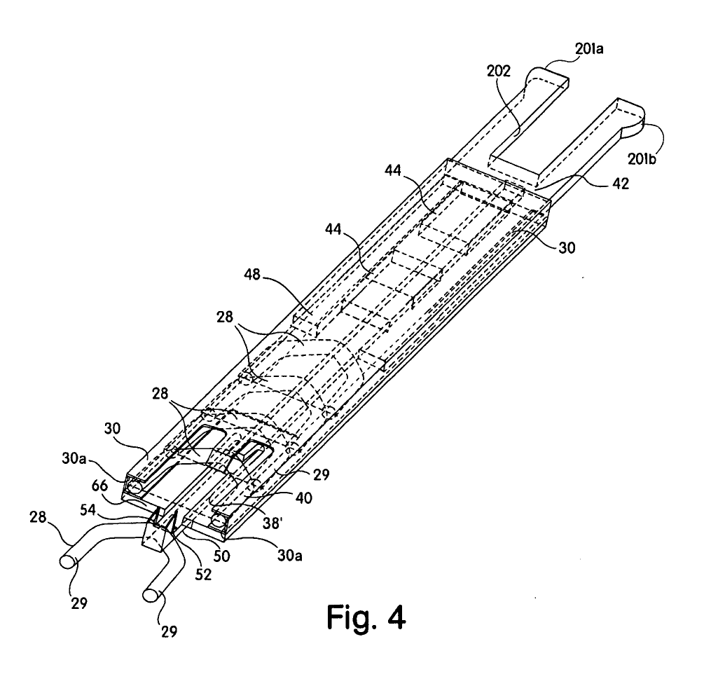

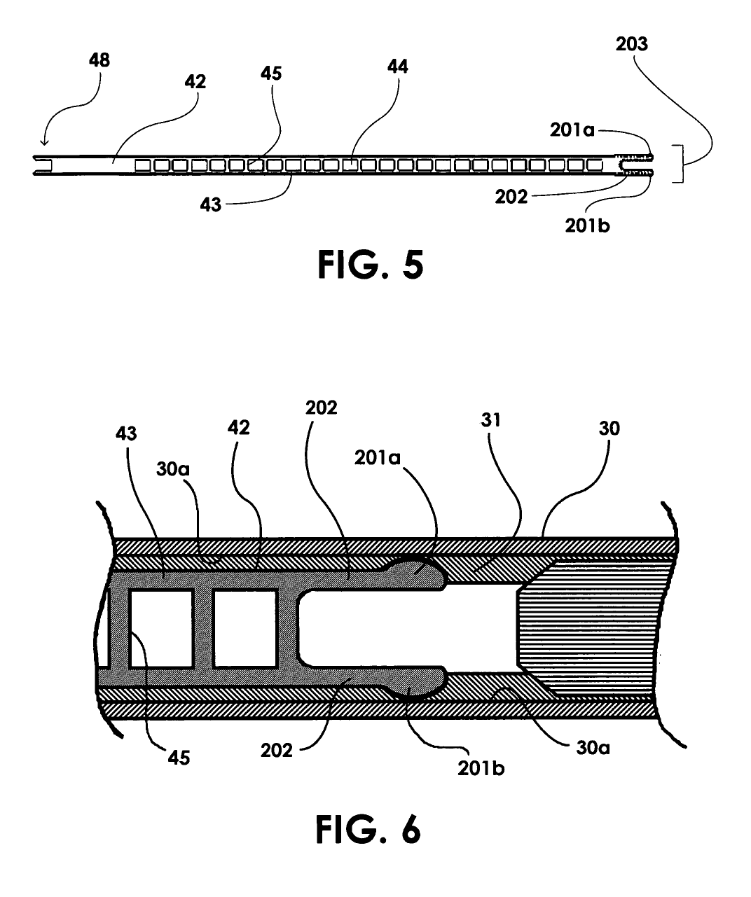

The ladder 42 may include first and second proximal protrusions 201 a and 201b (see FIG. 4, for example), which are flexibly and/or elastically biased outwardly away from the central longitudinal axis of the ladder 42. As shown in FIG. 5, for example, the ladder 42 may include a distal end having a clip abutment portion 48 which contacts the clip or clips 28 to urge the clips 28 distalward toward the jaws 12 for applying the clips 28 to the area to be clipped and/or stapled. The proximal end 203 of the ladder 42 may include the proximal protrusions 201a and/or 201b. Either one or both of the proximal protrusions (such as 201a and 201b) may extend outward and mutually away from each other, and there may be any appropriate number of protrusions (in other words, the number of protrusions is not necessarily limited to only two, as exemplified by the protrusions 201a and 201b in FIGS. 4 and 5, but may, as an alternative, include one protrusion, or three protrusions, or any other number of protrusions, for example).

When the ladder 42 is slidably disposed in the cartridge 30, and the cartridge 30 is inserted into the barrel 24, the outward protrusion or bias of the proximal protrusions 201 exert a force laterally against inner side walls 30a of the cartridge 30—i.e., laterally outward from a central lengthwise axis of the cartridge (as exemplified by line A-A in FIG. 10)—and thus a frictional force also results when the ladder 42 slides within the cartridge 30, for example. This frictional force may slow down the rate at which the ladder 42 slides relative to the cartridge 30 and barrel 24, and in particular—in a non-limiting example—this frictional force caused by the proximal protrusions 201 may beneficially inhibit (“slow down”) or prevent the ladder 42 from unintentionally pushing a clip 28 out of the distal tip of the clip applying device 14 when the ladder 42 slides distalward in the cartridge 30 as a result of gravity or inertia acting on the body of the ladder 42.

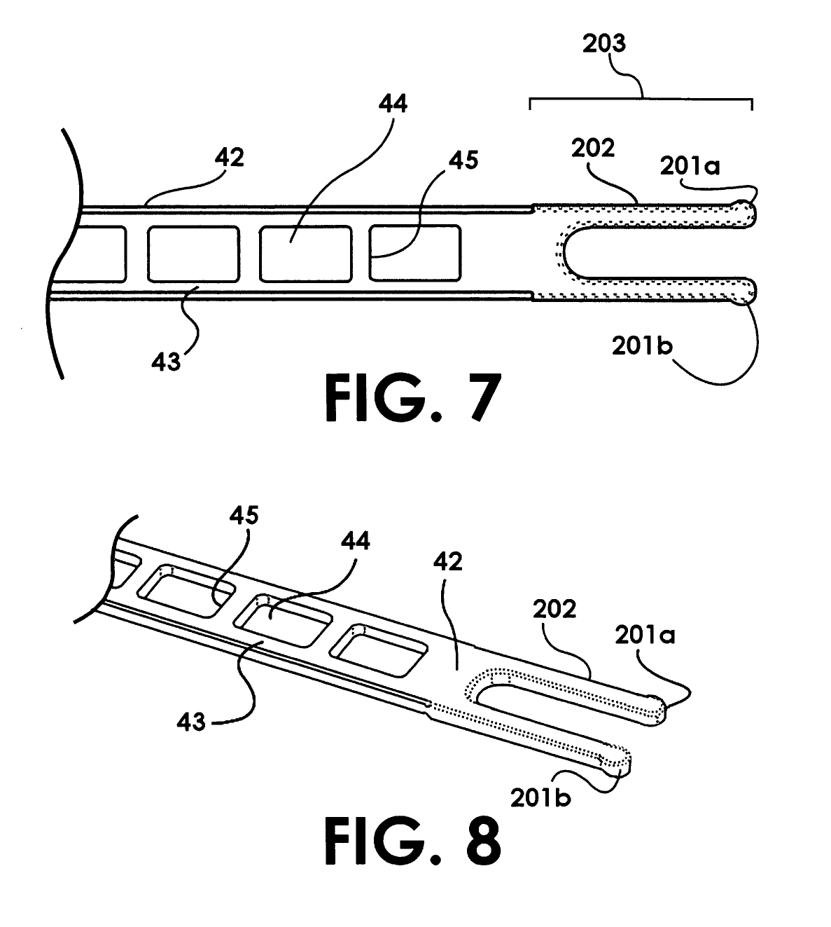

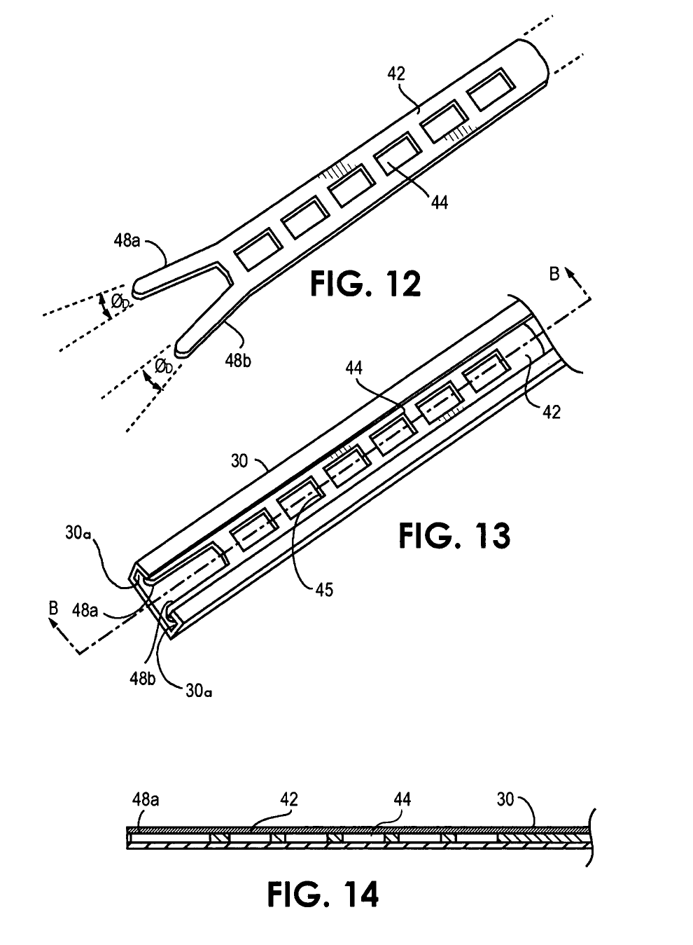

It is to be understood that the phrase “laterally protruding,” as generally used herein unless otherwise noted, refers to, for example, extension in a direction generally coplanar with the broadest plane of the ladder. For example, FIG. 12 shows two distal protrusions 48a and 48b which extend outward mutually away from one another (and each also extends away from the central axis of the ladder 42, as illustrated by the line B-B in FIG. 13) while coplanar with the flattest plane defined by the body of the ladder 42 (e.g., the plane formed by the side rails 43 and the cross rails 45, as shown in FIG. 7).

Furthermore, the terms “lateral” and “laterally” are generally understood to be considered relative to a reference object or reference direction, such as, for example, the flat plane of the ladder 42 (as discussed above, for example), and is used for convenience herein with respect to such reference object or reference member but is not in any way intended to be necessarily limited to an absolute or fixed geometrical frame of reference (although such a fixed frame of reference is also understood to fall within the use herein of the terms “lateral” and “laterally,” as such a situation can also properly correspond to aspects and/or embodiments of the present invention). Rather, it is to be understood that if a particular reference object, such as the ladder, corresponding to a use of the term “lateral” changes its geometrical position and/or orientation with respect to a local, area-wide and/or global frame of reference, and/or with respect to another object such as the rest of the clip applying device 14, if appropriate, that the term “lateral” is understood to still be properly interpreted with respect to the particular reference object, for example.

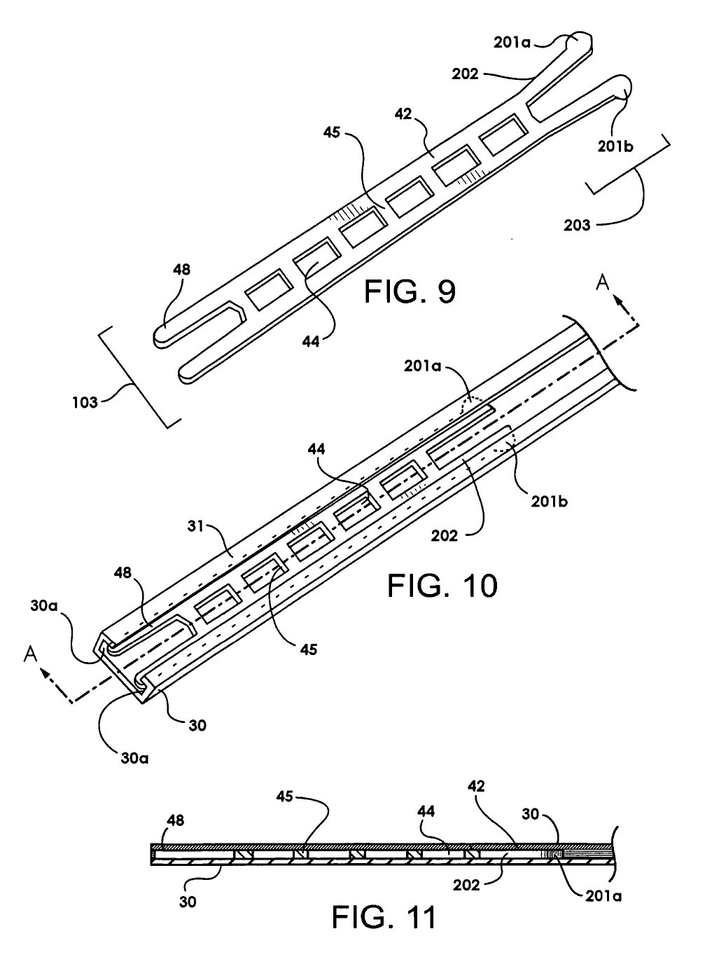

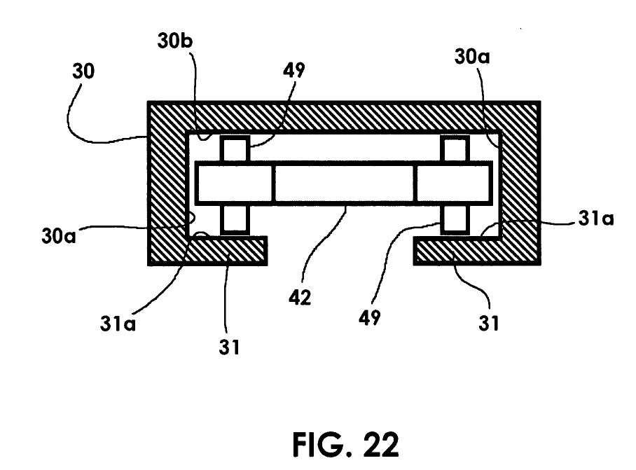

Also, it is further to be understood that the term “protrusion” as generally used herein is an inclusive term intended to denote any and all protruding portions, and is not intended to be limited to any particular example of a protrusion. For example, a “protrusion” can refer to either the rounded ends 201a and 201b, which extend laterally outward beyond the outermost lateral extent of the side rails 43 of the ladder 42, and/or may also refer to both the rounded ends 201a and 201b and the extension members 202 as shown in FIG. 9, either collectively or respectively, interalia. The term “protrusion” can also refer to a distal protrusion such as 48a as shown in FIG. 12, and/or a vertically extending protrusion such as an orthogonal vertical riser 49 exemplified in FIG. 22, and/or any other suitable protruding portion or member.

In some variations and/or embodiments of the present invention, the rounded protrusions (such as 201a and/or 201b) at the tip of the extension members 202 may be omitted; alternatively (or additionally), in some variations and/or embodiments, the extension members 202 may be generally straight and oriented generally parallel to the central axis of the ladder 42, with the rounded protrusions (such as 201a and/or 201b) extending outward (as illustrated in FIGS. 7 and 8, for example); and/or the extension members 202 may themselves protrude and/or skew outward with regard to the central axis of the ladder 42, as illustrated in FIG. 9, for example.

Furthermore, the frictional force generated by the proximal protrusions 201 abutting the cartridge 30 may be selected (by, for example, choosing the angle of protrusion of the proximal protrusions 201 and/or a shape of a frictional contact surface of the ladder 42, choosing a material having a particular elasticity, and/or choosing a material for the ladder 42 and/or cartridge 30 having a particular coefficient of friction—at least for the frictional surface which contacts a wall of the cartridge 30, inter alia) such that the frictional force is sufficient to produce the beneficial speed-reducing effect, but which nonetheless does not inhibit or suppress the intended functionality of the medical stapler 14 to apply a clip 28 to the jaws 12 when the trigger is operated by the user, for example.

FIG. 5 shows an example of a ladder 42 in accordance with the first embodiment, and which has two proximal protrusions 201a and 201b which are formed from the side rails of the ladder 42, and a distal end having a clip abutment portion 48 for pushing the clips 28 toward the distal end 26 of the barrel 24 (see FIG. 1, for example). As shown in FIG. 6, when the ladder 42 is slidably housed in the cartridge 30, the two contact surfaces at the proximal end of the proximal protrusions 201a and 201b abuts the side of the cartridge 30, in order to generate the frictional force therewith. FIGS. 7 and 8 also show the proximal end of an example of a ladder 42 according to the first embodiment, in which the proximal protrusions 201a and 201b have rounded frictional contact portions at their respective proximal ends.

Also, FIGS. 9, 10 and 11 show the effect of the proximal protrusions 201a and 201b when the ladder 42 is slidably housed within the cartridge 30. For example, although the ladder 42 has proximal protrusions 201a and 201b which are bent generally laterally outward away from the central axis of the ladder 42, once the ladder is inserted into the cartridge 30 the proximal protrusions 201a and 201b compress to a generally straight configuration (i.e., generally parallel and coaxial with the side rails of the ladder 42) while the elasticity of the ladder 42 urges the protrusions 201a and 201b against the side walls 30a of the cartridge 30 (see FIG. 10). However, as shown in FIG. 11, because the force exerted by the proximal protrusions 201a and 201b is directed laterally rather than along a vertical direction, the ladder 42 is therefore not warped along its length, which may be beneficial compared to the configuration shown in FIG. 25, for example.

As an alternative to having two protrusions, a ladder in accordance with a variation of the first embodiment (and/or other embodiments) may include one protrusion, for example (as discussed above). In a non-limiting example of such a variation of the first embodiment, a ladder having one protrusion may include the protrusion which laterally abuts an inner side surface of the cartridge (or any other appropriate surface of the cartridge), and opposite the protrusion the ladder may simply have a side rail or any other appropriate member. As the one protrusion abuts the cartridge, the side rail or other member which is positioned opposite the one protrusion may also be urged against the cartridge because the force applied by the one protrusion against the cartridge would be distributed between both the one protrusion and the side rail or other member positioned opposite the one protrusion. Therefore, a ladder having even one protrusion may provide elastic force in order to generate frictional contact (between the cartridge and either the protrusion or the side rail, or both, for example) between the ladder and the cartridge, for example.

In accordance with a second embodiment of the present invention, for example, the distal end 103 of the ladder 42 includes distal protrusions 48a and 48b extending from both side-rails of the ladder 42 (see FIGS. 12, 13 and 14, for example), which are elastically biased outward (that is, laterally) away from the center axis of the ladder 42 by the elasticity inherent in the material of which the ladder 42 is composed. As shown in FIG. 12, for example, the distal protrusions 48a and 48b may be naturally biased outward from the straight axes of both side-rails (the respective straight axes of which are parallel to one another) of the ladder 42 by an angle θ_{D}, such that when the ladder 42 is enclosed by the cartridge 30, the inner side walls 30a of the cartridge 30 elastically deform the distal protrusions 48a and 48b back toward the central axis of the ladder 42.

In such a state, the elasticity of the distal protrusions 48a and 48b (see FIG. 12) causes them to abut against the side walls 30a of the cartridge 30, and accordingly effects a frictional resistance therebetween in proportion with the elastic force exerted by the enclosed distal protrusions 48a and 48b and the coefficients of friction of the distal protrusions 48a and 48b against the side walls of the cartridge 30 (as illustrated in FIG. 13 and in a manner generally similar as discussed regarding the protrusions 201a and 201b of the first embodiment, for example). This frictional resistance may serve to slow or retard the sliding movement of the ladder 42 within the cartridge 30 and/or barrel 24; and therefore, inadvertent sliding of the ladder 42 may be reduced or eliminated (see FIG. 14), such that the unintentional release of clips from the clip device 14 is inhibited.

Moreover, because the distal protrusions 48a and 48b are biased away from the central axis of the ladder 42 generally only in the substantially flat lateral plane of the ladder 42, as a result little or no directional component of the force exerted by the elasticity of the distal protrusions 48a and 48b enclosed by the cartridge 30 is directed in a directional orthogonal to the plane of the ladder 42. Therefore, warping or bending of the ladder 42 along the length of the ladder 42 may be reduced or eliminated (as shown in FIG. 14, for example; compare with the configuration shown in FIG. 25, in which significant warping may occur).

The ladder 42 may be formed of a single material (plastic, polyvinyl chloride, polyethylene, polystyrene, polymer or any other suitable material, for example) therethroughout, by molding or casting, for example; or, alternatively, the distal protrusions 48a and 48b may otherwise be formed of a different material from the main body of the ladder 42 and later adhered or otherwise affixed to the ladder 42, for example.

Also, in an alternative configuration, the ladder 42 may include both proximal protrusions such as 201a and 201b at the proximal end 203 of the ladder 42, as shown in FIG. 9, as well as distal protrusions such as 48a and 48b at the distal end 103 of the ladder 42, as illustrated in FIG. 12, for example, and/or any combination thereof, rather than including only proximal or distal protrusions alone, for example.

In accordance with at least one variation of the second and/or other embodiments, the protrusions—such as, for example, either the proximal protrusions 201a and 201b or the distal protrusions 48a and 48b, or both—may be disposed at any point between the proximal end 203 and the distal end 103, for example.

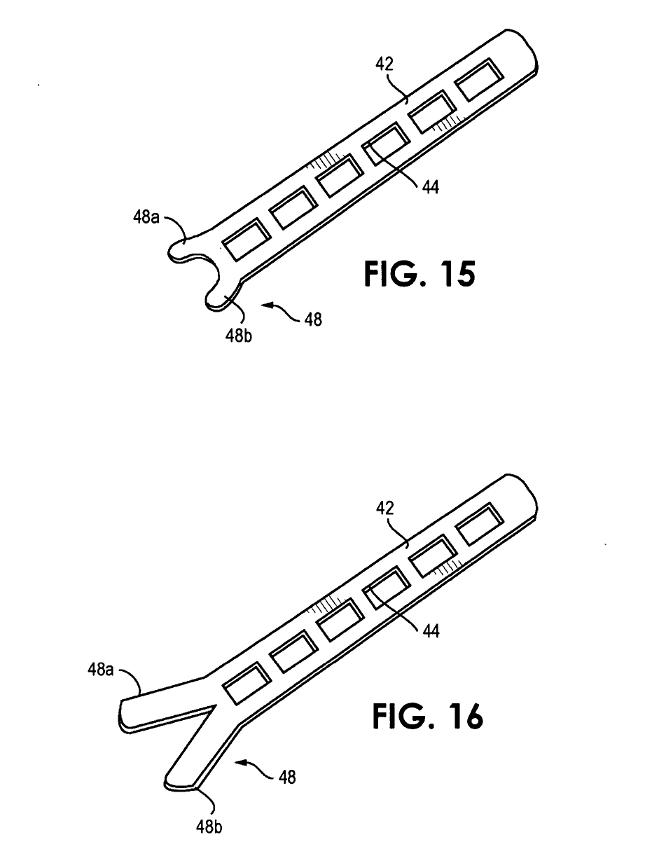

Referring to FIG. 15, for example, a third embodiment of the present invention is illustrated, in which the distal protrusions 48a and 48b of the ladder 42 are comparatively of shorter length and greater outward alignment than in the first embodiment (and may have a form similar to a letter ‘U,’ for example, as shown in FIG. 15). Because of further outward biasing (caused in part, for example, by the greater stubbiness of the general ‘U’ shape of the distal end 48) of the distal protrusions 48a and 48b, the central portion of the ladder 42 may be narrower than the first embodiment. As a result, it may be possible to reduce the overall mass and weight of the ladder 42, and possibly to obtain lower manufacture costs and a lower likelihood of inadvertent ladder sliding.

FIG. 16 also illustrates an alternative configuration of the third embodiment of the present invention, in which the distal protrusions 48a and 48b of the ladder 42 form a shape generally similar to a letter ‘V,’ for example, although various other shapes or forms may also be selected, such as a partial hexagonal shape similar to a wrench-head, for example, or any other suitable form.

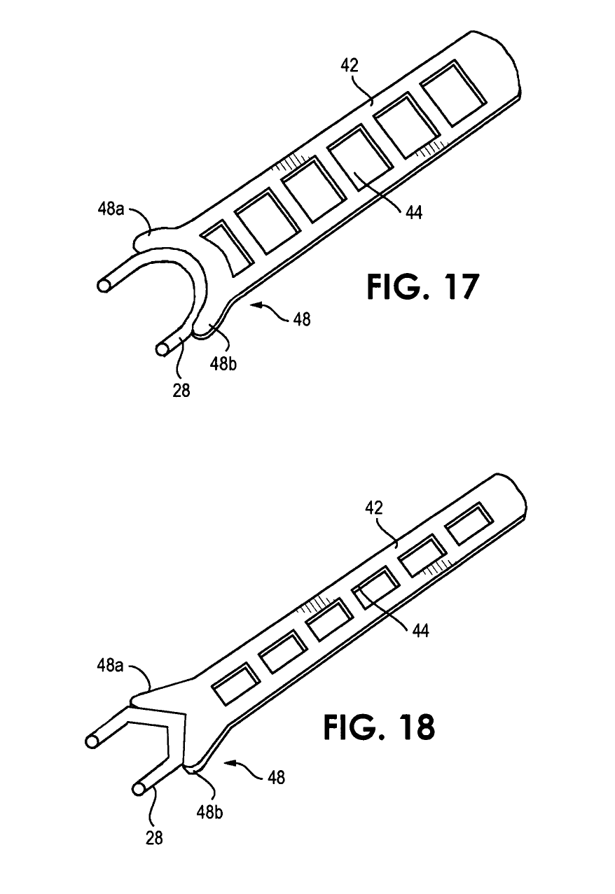

FIGS. 17 and 18 illustrate a fourth embodiment of the present invention, in which the end portion (including, in the exemplified embodiments, the distal protrusions 48a and 48b) of the ladder 42 forms an abutment portion and in which the clips 28 are designed having a form or shape that conforms to the abutment portion of the ladder 42. Examples of these are parabolic or semicircular shape, as shown in FIG. 9A, or an angled shape, as shown in FIG. 9B, for example (and similar to the exemplary shapes discussed above in regard to the third embodiment).

Because of the conformation of the clips 28 to the abutment portion of the ladder 42, the appropriate alignment of the clips 28 may be bolstered by the shape of the abutment portion of the ladder 42.

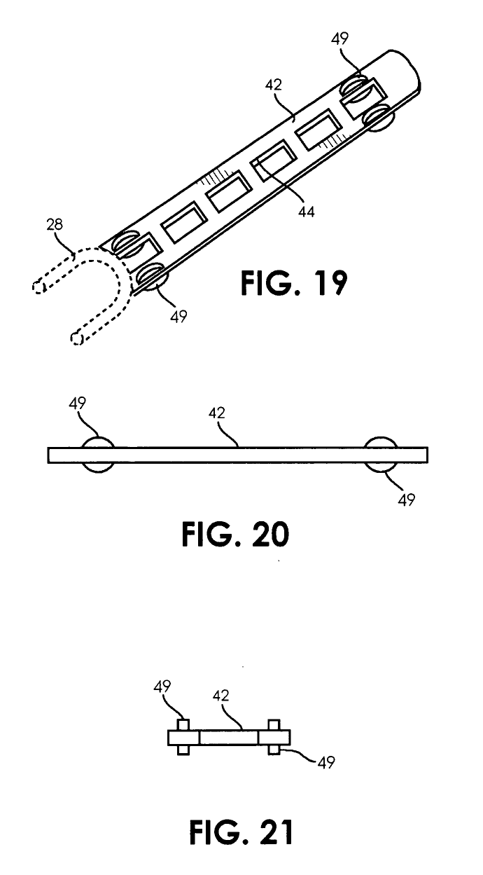

A fifth embodiment of the present invention is exemplified in FIGS. 19, 20, 21 and 22. In accordance with the fifth embodiment, as shown in FIG. 19, the ladder 42 may omit proximal protrusions 48a, 48b and/or distal protrusions 201a, 201b, (although omission is not necessary, and any combination of proximal and/or distal protrusions may also simultaneously be included), while providing the frictional contact between the ladder 42 and cartridge 30 via one or more orthogonal vertical risers 49 which abut inner surfaces 31a of respective flanges 31 of the cartridge 30 (see FIG. 22, for example) and/or the ceiling 30b (the term “ceiling” is understood to refer to a generally upper inner surface of a cartridge or other member, such as illustrated in a non-limiting example by reference number 30b in FIG. ) or floor of the cartridge 30 (similarly, the term “floor” is understood to refer to a generally lower inner surface of a cartridge or other member).

It is noted that as used herein, directional terms such as “upper” and “lower,” “upward” and “downward,” are generally to be interpreted relative to a reference object or reference frame, as appropriate, and are not necessarily limited to any particular global or fixed frame of reference (although such terms also not necessarily exclude such a global, area-wide, local, or fixed frame of reference, for example). It is further understood that such terms may be appropriately geometrically translated and/or interpreted relative to the appropriate reference object or reference frame, and thus may retain their proper interpretations, even if the reference frame or reference object changes its position and/or orientation relative any other particular object or frame; such directional terms are not intended as terms of limitation, but rather simply to exemplify such features as they describe.

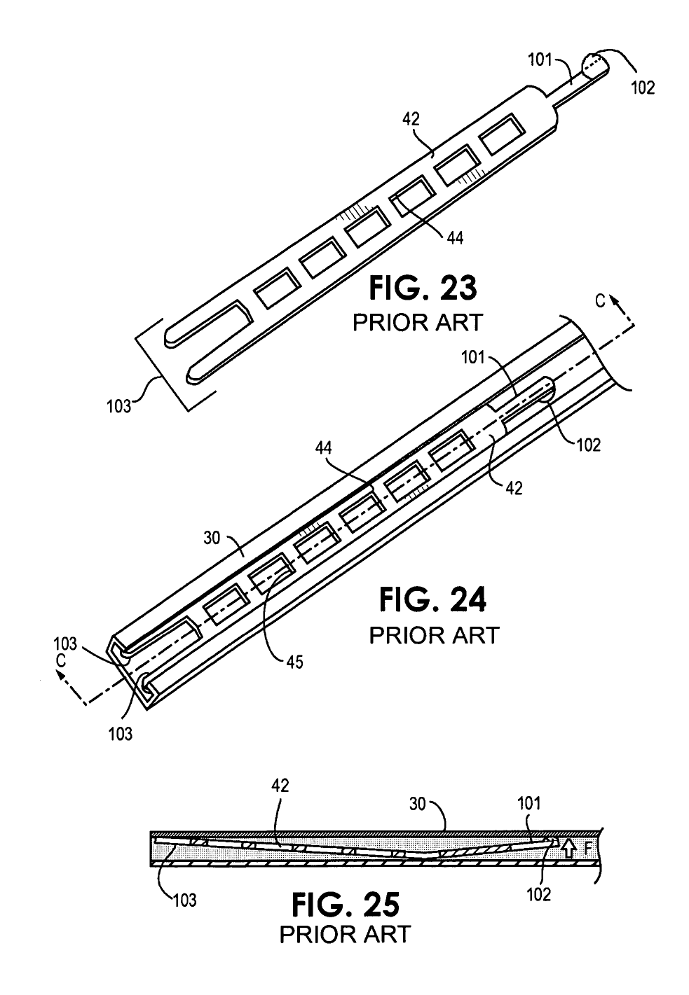

In contrast to the “finger” or “tail” configuration shown in FIGS. 23-25, the orthogonal vertical risers 49 are braced by virtue of their fin-like or boss-like shape, such that the downward force exerted by the risers 49 against the cartridge 30 is canceled by a corresponding upward force exerted by an orthogonal vertical riser 49 on the other side of the ladder 42, for example (the vertical risers 49 may have any appropriate shape or form, such as, for example, round, flat, oblong, peg-like, or square, but not necessarily limited thereto). Therefore, warping of the ladder 42 may be prevented while beneficial frictional force may be generated between the orthogonal vertical risers 49 and the cartridge 30 in order to retard the sliding movement of the ladder within the cartridge 30, for example.

For example, a ladder 42 as shown in FIG. 19 may include two orthogonal vertical risers 49, both extending from a position along a side rail 43 of the ladder 42. In this example, the first orthogonal vertical riser 49 extends from the top of the side rail 43 and the second orthogonal vertical riser extends from the bottom of the side rail 43, such that the reactive forces applied to each of the orthogonal vertical risers 49 when the ladder 42 is housed within a cartridge 30 (see FIG. 22) substantially cancel each other (and therefore apply approximately zero net torque to the ladder 42). In other words, if the respective reactive forces of the oppositely-disposed orthogonal vertical risers 49 were modeled by force vectors V₁ and V₂, in which V₁ corresponds to the reactive force applied to the top orthogonal vertical riser and V₂ corresponds to the reactive force applied to the bottom orthogonal vertical rider, and in which V₁ and V₂ include constituent force components in each of an X-axis direction, a Y-axis direction, and a Z-axis direction which are each mutually orthogonal, then the vector sum of V₁ and V₂ is zero (i.e., V₁+V₂=0).

As illustrated in FIG. 21, for example, the vertical risers 49 extending from the side rails 43 of the ladder 42 are oriented to extend in a direction generally orthogonal to the primary plane of the ladder 42 (that is, the plane defined generally by the side rails 43 and the cross rails 45 of the ladder 42). Conversely, FIGS. 7 and 8 illustrate the first embodiment in which the proximal protrusions 201a and 201b are oriented to extend laterally outward from the ladder 42 and are generally coplanar with the ladder 42 (i.e., are generally within—or at least parallel to—the primary plane of the ladder 42).

Also, the orthogonal vertical risers 49 may include substantially the a same material as the ladder 42, or may include a different material; furthermore, for example, the orthogonal vertical risers 49 may include an elastic member such as a spring (which also may be made of the same material the ladder 42 or of a different material, such as metal, for example).

In addition, the elasticity for providing the frictional force between the ladder 42 and the cartridge 30 may be inherent to the cartridge 30 and/or the ladder 42 (or its components, such as the orthogonal vertical risers 49, the distal or proximal protrusions 48a and 48b or 201a and 201b). For example, the orthogonal vertical risers 49 may be made of a generally rigid material, while the cartridge 30 may have a generally ‘C’-shaped cross-section and include a flexible metallic or polymer material, such that the upper lips of the cartridge 30 can elastically flex to accommodate the ladder 42 and orthogonal vertical risers 49. In such an arrangement, the elasticity of the cartridge 30 may provide the frictional force between the orthogonal vertical risers 49 and the cartridge 30. As a benefit, for example, the fabrication costs of the ladder 42 and/or cartridge 30, may be reduced.

It is noted that the foregoing examples have been provided merely for the purpose of explanation and are in no way to be construed as limiting of the present invention. While the present invention has been described with reference to certain embodiments, it is understood that the words which have been used herein are words of description and illustration, rather then words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A clip applying device, comprising:
a barrel having a first end and a second end;
a clip applicator affixed to the second end of the barrel and configured to apply a clip during a surgical procedure;
an actuation assembly attached to the first end of the barrel and configured to actuate the ladder and clip applicator when operated by a user;
a cartridge disposed within the barrel extending generally from the first end of the barrel to the second end of the barrel; and
a ladder slidably disposed within the cartridge and including a first protrusion extending laterally outward from a central axis of the ladder.

2. The clip applying device according to claim 1, further comprising:
a second protrusion extending laterally outward from a central axis of the ladder in a direction generally opposite from the first protrusion.

3. The clip applying device according to claim 2, wherein the cartridge has a cross-section generally similar to a letter ‘C’ and includes first and second inner side surfaces, and
wherein the first and second protrusions of the ladder frictionally abut the first and second inner side surfaces, respectively.

4. The clip applying device according to claim 1, wherein the ladder is substantially flat when disposed in the cartridge.

5. The clip applying device according to claim 1, wherein the ladder includes a clip abutment portion including the first protrusion and configured to abut a first clip nearest the actuation assembly among a plurality of clips, and
wherein the first clip has a shape configured to conform to the clip abutment portion of the ladder.

6. The clip applying device according to claim 1, wherein the cartridge is insertable into and removable from the clip applying device,
wherein the cartridge houses the ladder therein, and
wherein the cartridge is configured to contain a plurality of clips and to be inserted into the first end of the barrel.

7. The clip applying device according to claim 1, wherein the first protrusion extends from at least one of a distal end of the ladder farthest from the actuation assembly, a proximal end of the ladder nearest to the actuation assembly, or a point between the proximal and distal ends.

8. A clip applying device, comprising:
a barrel having a first end and a second end;
a clip applicator affixed to the second end of the barrel and configured to apply a clip during a surgical procedure;
an actuation assembly attached to the first end of the barrel and configured to actuate the ladder and clip applicator when operated by a user;
a cartridge disposed within the barrel extending generally from the first end of the barrel to the second end of the barrel; and
a ladder slidably disposed within the cartridge and including a side rail extending longitudinally in the cartridge, a first protrusion extending from a top of the rail and a second protrusion extending from a bottom of the rail at a position opposite the first protrusion, the first and second protrusions configured to abut the cartridge.

9. The clip applying device according to claim 8, wherein the cartridge has a cross-section generally similar to a letter ‘C’ and includes a lower inner surface and an upper inner surface generally parallel to the bottom surface, and
wherein the first and second protrusions of the ladder frictionally abut the lower inner surface and the upper inner surface, respectively.

10. The clip applying device according to claim 8, wherein the first protrusion exerts a downward force and the second protrusion exerts an upward force, and
wherein the upward force and the downward force impart generally zero net torque to the ladder.

11. The clip applying device according to claim 8, wherein the ladder is substantially flat when disposed in the cartridge.

12. The clip applying device according to claim 8, wherein the ladder includes a clip abutment portion configured to abut a first clip nearest the actuation assembly among a plurality of clips, and
wherein the first clip has a shape configured to conform to the clip abutment portion of the ladder.

13. The clip applying device according to claim 8, wherein the cartridge is insertable into and removable from the clip applying device,
wherein the cartridge houses the ladder therein, and
wherein the cartridge is configured to contain a plurality of clips and to be inserted into the first end of the barrel.

14. The clip applying device according to claim 8, wherein the first and second protrusions extend from at least one of a distal end of the ladder farthest from the actuation assembly, a proximal end of the ladder nearest to the actuation assembly, or a point between the proximal and distal ends.

15. A ladder for use with a cartridge for a clip applying device, the ladder comprising:
first and second protrusions extending from an end of the ladder along at least one of a lateral plane generally coplanar with the ladder or an orthogonal plane generally orthogonal to the ladder, the ladder configured to serially urge a clip outward from the clip applying device,
wherein the first and second protrusions are configured to abut a cartridge in which the ladder is disposed.

16. The ladder according to claim 15, wherein the ladder is disposed in a cartridge configured to engage a barrel of the surgical stapler, wherein the cartridge has a cross-section generally similar to a letter ‘C’ and includes first and second inner side surfaces, and
wherein the first and second protrusions of the ladder frictionally abut the first and second side surfaces, respectively.

17. The ladder according to claim 15, further comprising a plastic material.

18. The ladder according to claim 15, wherein the ladder is disposed in a cartridge configured to engage a barrel of the surgical stapler, wherein the cartridge has a cross-section generally similar to a letter ‘C’ and includes an inner bottom surface and an inner upper surface, and
wherein the first and second protrusions of the ladder include vertical risers configured to frictionally abut the inner bottom surface and the inner upper surface, respectively.

19. The ladder according to claim 15, further comprising a clip abutment portion including the first and second protrusions, and
wherein clip abutment portion has a generally parabolic shape configured to conform to the clip.

20. The ladder according to claim 15, further comprising third and fourth protrusions extending from an end of the ladder generally opposite the first and second protrusions and configured to abut the cartridge in which the ladder is disposed.
